(19) United States
(12) Patent Application Publication
(10) Pub. No.: US 2026/0240799 A1
(43) Pub. Date: Aug. 20, 2026

(54) TREATMENT OF PRIMARY BILIARY CHOLANGITIS

(71) Applicant: Genfit, Loos (FR)

(72) Inventors: Pascal Birman, Paris (FR); Julie Dietrich, Boston, MA (US)

(21) Appl. No.: 19/643,940

(22) Filed: Apr. 10, 2026

Related U.S. Application Data
(63) Continuation of application No. PCT/EP2024/078822, filed on Oct. 13, 2024.

(30) Foreign Application Priority Data
Oct. 13, 2023 (EP) 23306800.6

(51) Int. Cl. A61K 31/192; A61K 31/575; A61P 1/16; A61P 43/00

(52) U.S. Cl. CPC A61K 31/192; A61K 31/575; A61P 1/16; A61P 43/00

(57) ABSTRACT

The present invention relates to a pharmaceutical composition comprising a compound selected from the group consisting of elafibranor, 2-[2,6-dimethyl-4-[3-[4-(methylthio)phenyl]-3-oxo-propyl]phenoxy]-2-methylpropanoic acid (GFT1007), and a pharmaceutically acceptable salt of elafibranor or GFT1007, for use in a method for reducing fatigue in subject having primary biliary cholangitis (PBC). The pharmaceutical composition of the invention provides a significative reduction of the PROMIS Fatigue T-score (PROMIS Short Form—Fatigue 7a questionnaire (V1.0)) from baseline in a subject having PBC.

### RELATED APPLICATIONS

This application is a Continuation of International Application No. PCT/EP2024/078822, filed Oct. 13, 2024, which claims the benefit of and priority to European Application No. 23306800.6, filed Oct. 13, 2023. The entire teachings of all of the above applications are incorporated herein by reference. International Application No. PCT/EP2024/078822 was published under PCT Article 21(2) in English.

### TECHNICAL FIELD

The present invention relates to the field of medicine, in particular the treatment of cholestatic diseases, and more specifically PBC.

### BACKGROUND

Primary biliary cholangitis (PBC) is a rare, chronic, progressive, cholestatic liver disease of autoimmune etiology, characterized by injury of the intrahepatic bile ducts that, in untreated patients, may progress to hepatic fibrosis, cirrhosis, hepatic decompensation, and death unless patients receive a liver transplant. PBC disproportionately affects women vs men (approximately 10:1) and is typically diagnosed in patients between 40 years to 60 years of age. In Europe, North America, Asia, and Australia, the incidence and prevalence rates of PBC are reported as ranging from 0.33 to 5.8 per 100,000 inhabitants and 1.91 to 40.2 per 100,000 inhabitants, respectively.

Over 60% of the newly diagnosed cases are asymptomatic. The majority of asymptomatic patients become symptomatic within 10 years.

PBC represents one of the leading indications for liver transplantation. Despite its rarity, PBC remains therefore an important cause of morbidity in the Western world. PBC has also been identified as an important risk factor for hepatocellular carcinoma.

PBC is characterized by cholestasis caused by autoimmune destruction of biliary ductules with progressive impairment of bile flow in the liver. This results in increased hepatocellular bile acid concentrations which are toxic to the liver. Such hepatocellular injury is associated with a local inflammatory response resulting early on in an abnormal elevation of serum alkaline phosphatase (ALP) levels. Indeed, elevations in ALP level are associated with a risk of liver transplantation or death that is 2.0 to 2.5 times higher than the risk associated with normal levels. An abnormally elevated bilirubin level, which occurs later in disease progression, is also a strong predictor of outcomes, with a risk of liver transplantation or death that was 5.1 to 10.7 times the risk associated with normal levels.

Fatigue is considered one of the most frequent and debilitating symptoms in primary biliary cholangitis (PBC), affecting over 50% of PBC patients. One in five patients with PBC suffer from severe fatigue, which significantly impairs quality of life. Fatigue is made up of a central and a peripheral component, whose pathophysiology is still greatly unresolved (E. N. Lynch et al.; World J Hepatol. 2022 Jun. 27; 14(6): 1111-1119). As opposed to itching, and with the exception of end-stage liver disease, fatigue is not related to disease progression.

In October 2023, the only approved drugs to treat patients with PBC were ursodeoxycholic acid (UDCA) and Ocaliva® (obeticholic acid, OCA).

UDCA (Ursodeoxycholic acid) has been shown to improve ALP and bilirubin levels, and to delay histological progression, thereby increasing liver transplant-free survival. However, up to 40% of UDCA-treated patients have a suboptimal response (Ali A H, et al., Orphan drugs in development for primary biliary cirrhosis: challenges and progress. Orphan Drugs: Research and Reviews. 2015; 5:83-97). ALP has been shown to remain elevated in up to 70% of patients who are currently being treated or are intolerant to UDCA (Lammers W J, et al., Levels of alkaline phosphatase and bilirubin are surrogate end points of outcomes of patients with primary biliary cirrhosis: an international follow-up study. Gastroenterology. 2014; 147(6):1338-1349.). Such patients remain at risk of disease progression and longer term adverse clinical outcomes. At present, 44% of UDCA-treated patients are progressing to liver transplant or death over 15 years.

Obeticholic acid (OCA) (Ocaliva®), which has been shown to reduce ALP, has been recently approved in several countries as second line therapy for the treatment of PBC as monotherapy in adults unable to tolerate UDCA or in combination with UDCA in adults with an inadequate response to UDCA. A decrease in ALP levels is recognized as a particularly relevant surrogate marker for the treatment of PBC, and was recently used as the basis for conditional market approval of OCA in this indication.

However, fatigue is not responsive to UDCA or OCA therapy. Fatigue is even defined as one of the most common side effects of OCA treatment. To date, there is no effective therapy for fatigue caused by PBC, and therefore, the approach to fatigue and its management needs to run in parallel with management of the underlying disease.

Considering the efficacy and tolerability issues with the current treatment options available, there is an unmet need for therapeutic options for patients with PBC, allowing a significative reduction of fatigue.

Elafibranor (2-(2,6-dimethyl-4-{3-[4-(methylsulfanyl)phenyl]-3-oxopropen-1-yl}phenoxy)-2-methylpropanoic acid) was evaluated in a phase 3 study for the treatment of PBC (ELATIVE™) The results of phase 3 on PBC show that the mean relative change (%) from baseline to endpoint in serum ALP was −48.3% for the elafibranor 80 mg treatment group, and 3.2% for placebo.

Thus, the treatment with elafibranor resulted in a consistent, statistically significant reduction in plasma ALP levels from baseline when compared to placebo. Moreover, elafibranor is safe and well-tolerated by the patients.

### SUMMARY OF THE INVENTION

The phase 3 clinical study has surprisingly shown that the treatment of participants with 2-(2,6-dimethyl-4-{3-[4-(methylsulfanyl)phenyl]-3-oxoprop-1-en-1-yl}phenoxy)-2-methylpropanoic acid (elafibranor, formerly called GFT505) provides a significative reduction of the PROMIS Fatigue T-score (PROMIS® Short Form—Fatigue 7a (V1.0)) from baseline in participant treated with elafibranor 80 mg daily compared to participant with placebo.

The present invention thus relates to a pharmaceutical composition comprising a compound selected from the group consisting of elafibranor, 2-[2,6-dimethyl-4-[3-[4-(methylthio)phenyl]-3-oxo-propyl]phenoxy]-2-methylpropanoic acid (GFT1007), and a pharmaceutically acceptable salt of elafibranor or GFT1007, for use in a method for reducing fatigue in a subject having primary biliary cholangitis (PBC).

The present invention also relates to the use of a pharmaceutical composition comprising a compound selected from the group consisting of elafibranor, 2-[2,6-dimethyl-4-[3-[4-(methylthio)phenyl]-3-oxo-propyl]phenoxy]-2-methylpropanoic acid (GFT1007), and a pharmaceutically acceptable salt of elafibranor or GFT1007, for the manufacture of a medicament for use in a method for reducing fatigue in a subject having primary biliary cholangitis (PBC).

The present invention also relates to a method for reducing fatigue in a subject having primary biliary cholangitis (PBC), comprising administering to said subject, a therapeutic effective amount of a pharmaceutical composition comprising a compound selected from the group consisting of elafibranor, 2-[2,6-dimethyl-4-[3-[4-(methylthio)phenyl]-3-oxo-propyl]phenoxy]-2-methylpropanoic acid (GFT1007), and a pharmaceutically acceptable salt of elafibranor or GFT1007.

### DESCRIPTION OF THE FIGURES

FIGS. 1 and 2: Mean changes from baseline in PROMIS Fatigue T-score over time

FIG. 1 compares mean changes from baseline in PROMIS Fatigue T-score between participants having PBC under elafibranor treatment (80 mg daily) and participants in the placebo group from week 4 to week 52.

FIG. 2 compares mean changes from baseline in PROMIS Fatigue T-score between participants having PBC and pruritus under elafibranor treatment (80 mg daily) and placebo group from week 4 to week 52.

### DETAILED DESCRIPTION OF THE INVENTION

The present invention relates to a pharmaceutical composition comprising a compound selected from the group consisting of elafibranor, GFT1007, and a pharmaceutically acceptable salt of elafibranor or GFT1007, for use in a method for reducing fatigue in a subject having PBC.

The present invention also relates to a pharmaceutical composition comprising a compound selected from the group consisting of elafibranor, GFT1007, and a pharmaceutically acceptable salt of elafibranor or GFT1007, for use in a method for reducing fatigue associated to PBC in a subject having PBC.

The present invention also relates to a pharmaceutical composition comprising a compound selected from the group consisting of elafibranor, GFT1007, and a pharmaceutically acceptable salt of elafibranor or GFT1007, for use in a method for treating PBC in a subject having PBC with fatigue, without provoking and/or worsening the fatigue of said subject.

The present invention further relates to the use of a pharmaceutical composition comprising a compound selected from the group consisting of elafibranor, GFT1007, and a pharmaceutically acceptable salt of elafibranor or GFT1007, for the manufacture of a medicament for use in a method for reducing fatigue in a subject having PBC.

The present invention further relates to the use of a pharmaceutical composition comprising a compound selected from the group consisting of elafibranor, GFT1007, and a pharmaceutically acceptable salt of elafibranor or GFT1007, for the manufacture of a medicament for use in a method for reducing fatigue associated to PBC in a subject having PBC.

The present invention further relates to the use of a pharmaceutical composition comprising a compound selected from the group consisting of elafibranor, GFT1007, and a pharmaceutically acceptable salt of elafibranor or GFT1007, for the manufacture of a medicament for use in a method for treating PBC in a subject having PBC with fatigue, without provoking and/or worsening the fatigue of said subject.

The present invention also relates to a method for reducing fatigue in a subject having PBC, comprising administering to said subject, a therapeutic effective amount of a pharmaceutical composition comprising a compound selected from the group consisting of elafibranor, GFT1007, and a pharmaceutically acceptable salt of elafibranor or GFT1007.

The present invention also relates to a method for reducing fatigue associated to PBC in a subject having PBC, comprising administering to said subject, a therapeutic effective amount of a pharmaceutical composition comprising a compound selected from the group consisting of elafibranor, GFT1007, and a pharmaceutically acceptable salt of elafibranor or GFT1007.

The present invention also relates to a method for treating PBC in a subject having PBC with fatigue, without provoking and/or worsening the fatigue of said subject, comprising administering to said subject, a therapeutic effective amount of a pharmaceutical composition comprising a compound selected from the group consisting of elafibranor, GFT1007, and a pharmaceutically acceptable salt of elafibranor or GFT1007.

In the context of the invention, the terms “reducing fatigue” can refer to the decrease in the feeling of tiredness or sense of exhaustion. It can also refer to an increase in the sense of energy and in the ability to execute daily activities and function normally in family or social roles.

The patient's fatigue can be measured using the PROMIS Short Form—Fatigue 7a questionnaire (V1.0) to assess change from baseline in fatigue during treatment. The PROMIS Fatigue item banks assess a range of self-reported symptoms over the past seven days, from mild subjective feelings of tiredness to an overwhelming, debilitating, and sustained sense of exhaustion that likely decreases one's ability to execute daily activities and function normally in family or social roles. Fatigue is divided into the experience of fatigue (frequency, duration, and intensity) and the impact of fatigue on physical, mental, and social activities. PROMIS instruments are scored using item-level calibrations. The final score is represented by the T-score, a standardized score with a mean of 50 and a standard deviation (SD) of 10. A 1-point decrease of the PROMIS Fatigue T-score represents a meaningful change in fatigue. This improvement corresponds to changes in alertness and well-being that are easily noticeable. Likewise, a noticeable worsening corresponds to a 1-point increase of the PROMIS Fatigue T-score (Schattenberg et al.; “Evaluating Pruritus and Fatigue in Patients with Treatment-Refractory Primary Biliary Cholangitis” EASL 21-24 Jun. 2023).

The patient's fatigue can also be measured using the PBC-40 total fatigue score. The PBC-40, consisting of 40 questions distributed across six domains: fatigue, emotional, social, cognitive function, general symptoms, and itch.

In a specific embodiment, treatment with the pharmaceutical composition of the invention results in a decrease in the PROMIS Fatigue T-score of at least 1 point from baseline in patient having PBC. Preferably, treatment with the pharmaceutical composition of the invention results in a decrease in the PROMIS Fatigue T-score of at least 1.2 points, more preferably at least 1.4 points, from baseline in patient having PBC.

In another specific embodiment, treatment with the pharmaceutical composition of the invention results in a decrease in the PROMIS Fatigue T-score of at least 2 points from baseline in patient having PBC and pruritus. Preferably, treatment with the pharmaceutical composition of the invention results in a decrease in the PROMIS Fatigue T-score of at least 2.2 points, more preferably at least 2.3 points, from baseline in patient having PBC and pruritus.

In particular, the improvement in the PROMIS Fatigue T-score can be observed from 4 weeks of treatment with the pharmaceutical composition of the invention. The improvement in the PROMIS Fatigue T-score can be even greater from 13 weeks of treatment with the pharmaceutical composition of the invention. The improvement in the PROMIS Fatigue T-score can be even greater from 52 weeks of treatment with the pharmaceutical composition of the invention.

The term “treatment” or “treating” refers to therapy, prevention, or prophylaxis of PBC in a subject in need thereof. The treatment involves the administration of elafibranor, GFT1007 or a pharmaceutically acceptable salt thereof (such as via the administration of a pharmaceutical composition comprising elafibranor, GFT1007 or a pharmaceutically acceptable salt thereof) to a subject (e.g. a patient) having a declared disease to prevent, cure, delay, reverse, or slow down the progression of the disease, improving thereby the condition of patients. A treatment may be also administered to subjects that are either healthy or at risk of developing PBC.

The term “patient”, “subject” or “individual” are interchangeable and refer to a mammal and more particularly a human including adult and child. In the context of the invention, the patient is suffering from PBC or from PBC with pruritus. The subjects to be treated according to the invention can be appropriately selected on the basis of several criteria associated with cholestatic pathological processes such as previous and/or present drug treatments, associated pathologies, genotype, exposure to risk factors, as well as any other relevant biomarker that can be evaluated by means of any suitable immunological, biochemical, or enzymatic method. The subject to be treated is with PBC or PBC with pruritus, as characterized as follows:
the presence of at least 2 of the following 3 diagnostic factors:
   (i) history of elevated ALP levels for at least 6 months prior to Day 0 (randomization visit)
   (ii) positive Anti-Mitochondrial Antibodies (AMA) titers (> 1/40 on immunofluorescence or M2 positive by enzyme-linked immunosorbent assay (ELISA) or positive PBC-specific antinuclear antibodies
   (iii) liver biopsy consistent with PBC
ALP≥1.67× upper limit of normal (ULN)
optionally, taking UDCA for at least 12 months (stable dose for ≥6 months) prior to screening visit.

In some embodiments of the invention, GFT1007, the active metabolite of elafibranor, is used. GFT1007 is 2-[2,6-dimethyl-4-[3-[4-(methylthio)phenyl]-3-oxo-propyl]phenoxy]-2-methylpropanoic acid. Its properties and synthesis were described in PCT application WO2007/147879, where it is referred to as compound 1.

According to the present invention, the pharmaceutical composition of the invention may include a stereoisomer of elafibranor, of GFT1007, or of a pharmaceutically acceptable salt of elafibranor or of GFT1007.

A stereoisomer is an isomeric compound that has the same molecular formula and sequence of bonded atoms, but differs in the 3D-dimensional orientations of its atoms in space. The stereoisomers include enantiomers, diastereoisomers, cis-trans and E-Z isomers, conformers and tautomers.

Elafibranor or GFT1007 can be formulated as pharmaceutically acceptable salt, particularly an acid or base salt compatible with pharmaceutical use. Salts of elafibranor or GFT1007 implemented herein include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified compound.

In particular, “pharmaceutically acceptable salts” include inorganic as well as organic acids salts. Counter-ions may be selected from the following the non-exhaustive list: ammonia, L-arginine, benethamine, benzathine, tert-butylamine (erbumine), calcium hydroxide, choline hydroxide, deanol, diethanolamine (2,2′-iminobis(ethanol), diethylamine, epolamine (1-(2-hydroxyethyl)pyrrolidine), 2-(diethylamino)-ethanol, ethanolamine (2-aminoethanol), ethylenediamine, glycine, hydrabamine, 1H-imidazole, L-Lysine, magnesium hydroxide, meglumine (N-methyl-glucamine), 4-(2-hydroxyethyl)-morpholine, piperazine, potassium hydroxide, sodium hydroxide, triethanolamine (2,2′,2″-nitrilo-tris(ethanol)), tromethamine, zinc hydroxide, in particular tromethamine, potassium, sodium, benethamine, benzathine, L-arginine, ethanolamine, meglumine, glycine, erbumine, L-lysine, epolamine, choline, preferably tromethamine, potassium, sodium, benethamine, benzathine, L-arginine, more preferably tromethamine, potassium, sodium, L-arginine, more particularly tromethamine.

In particular embodiments, the invention implements an ammonia, L-arginine, benethamine, benzathine, tert-butylamine (erbumine), calcium, choline, deanol, diethanolamine (2,2′-iminobis(ethanol), diethylamine, epolamine (1-(2-hydroxyethyl)pyrrolidine), 2-(diethylamino)-ethanol, ethanolamine (2-aminoethanol), ethylenediamine, glycine, hydrabamine, 1H-imidazole, L-Lysine, magnesium, meglumine (N-methyl-glucamine), 4-(2-hydroxyethyl)-morpholine, piperazine, potassium, sodium, triethanolamine (2,2′,2″-nitrilo-tris(ethanol)), tromethamine or zinc salt of elafibranor or GFT1007. In a further particular embodiment, the salt of elafibranor or GFT1007 is selected from a tromethamine, potassium, sodium, L-arginine, benethamine, benzathine, ethanolamine, meglumine, glycine, erbumine, L-lysine, choline, epolamine, magnesium or 2-amino-2-methyl-propan-1-ol salt of elafibranor or GFT1007.

In a preferred embodiment, the pharmaceutical composition of the invention comprises elafibranor or a pharmaceutically acceptable salt thereof.

In a most preferred embodiment, the pharmaceutical composition of the invention comprises elafibranor.

Pharmaceutical composition used in the invention can comprise one or several excipients or vehicles, acceptable within a pharmaceutical context (e.g. saline solutions, physiological solutions, isotonic solutions, etc., compatible with pharmaceutical usage and well-known by one of ordinary skill in the art). This composition can also comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc. Elafibranor or GFT1007 can be formulated for enteral or parenteral administration. For example, elafibranor or GFT1007 can be formulated for oral, intravascular (e.g. intravenous or intra-arterial), intramuscular, intraperitoneal, subcutaneous, transdermal or nasal administration. The pharmaceutical composition can be a solid or liquid dosage form. Illustrative formulations include, without limitation, an injectable suspension, or suspension for oral ingestion, a gel, an oil, a pill, a tablet, a suppository, a powder, a gel cap, a capsule, an aerosol, an oinment, a cream, a patch, or means of galenic forms for a prolonged and/or slow release.

In a preferred embodiment, the pharmaceutical composition of the invention is administered orally. Preferably, the composition is formulated in a form selected from the group consisting of a gel, an oil, a pill, a tablet, a powder, a gel cap, a capsule, and a galenic form or device assuring a prolonged and/or slow release.

In a preferred embodiment, the pharmaceutical composition of the invention is formulated in a tablet.

As used herein, the term “therapeutically effective amount” refers to a quantity of elafibranor or GFT1007 which prevents, removes or reduces PBC and one of its adverse events, in particular a quantity of elafibranor or GFT1007 which prevents, removes or reduces PBC and fatigue. In particular, the amount of pharmaceutical salt of elafibranor or GFT1007 is intended as the amount of free form of elafibranor or GFT1007 in this pharmaceutical salt.

The quantity to be administered can be adapted by a person skilled in the art. In particular, doses and regimen of administration may be function of the stage and of the severity of PBC and/or fatigue to be treated, as well as of the weight, the age and the global health of the subject to be treated, as well as of the judgment of the doctor.

In a particular embodiment, elafibranor, GFT1007, or a pharmaceutically acceptable salt of elafibranor or of GFT1007 is administered at a dose varying between 10 mg and 200 mg per administration, preferentially between 80 mg and 120 mg per administration. In a further particular embodiment, elafibranor, GFT1007, or a pharmaceutically acceptable salt of elafibranor or of GFT1007 is administered at a dose of 80 mg per administration. In another particular embodiment, elafibranor, GFT1007 or a pharmaceutically acceptable salt of elafibranor or of GFT1007 is administered at a dose of 120 mg per administration.

Preferably, the pharmaceutical composition of the invention is administered once a day, in particular is orally administered once a day.

According to an embodiment, the pharmaceutical composition is a solid dosage form, such as a tablet. In a further particular embodiment, said tablet comprises between 10 mg and 200 mg of elafibranor, GFT1007, or of a pharmaceutically acceptable salt of elafibranor or of GFT1007, such as between 80 mg and 120 mg of elafibranor, GFT1007, or of a pharmaceutical salt of elafibranor or of GFT1007. For example, a tablet may comprise 80 mg of elafibranor or GFT1007 or a pharmaceutical salt of elafibranor or of GFT1007 or 120 mg of elafibranor or GFT1007 or a pharmaceutical salt of elafibranor or of GFT1007.

In yet another embodiment, a tablet comprising 80 mg of elafibranor or GFT1007 is orally administered once a day.

In yet another embodiment, a tablet comprising 120 mg of elafibranor or GFT1007 is orally administered once a day.

In a particular embodiment, the patient has PBC and responds at least partly to ursodeoxycholic acid (UDCA). In another embodiment, the patient having PBC has an inadequate response to ursodeoxycholic acid (UDCA).

In a particular embodiment, the invention relates to the use of the pharmaceutical composition comprising a compound selected from elafibranor, GFT1007, and a pharmaceutically acceptable salt of elafibranor or GFT1007, in combination with at least one other therapeutically active agent, in the method for reducing fatigue in patient having PBC. The other active agent may in particular be selected from other anti-cholestatic agents such as UDCA or OCA. The invention thus also relates to the combination of the pharmaceutical composition of the invention with UDCA or OCA, preferably UDCA. In particular, the method comprises administering elafibranor at a dose of 80 mg per administration and UDCA, to the subject in need thereof. More particularly, the method comprises administering elafibranor at a dose of 80 mg per administration and UDCA, to a subject having PBC and an inadequate response to ursodeoxycholic acid. Even more particularly, the method comprises administering elafibranor at a dose of 80 mg per day and UDCA at a dose of 13-15 mg/kg/day, to a subject having PBC and an inadequate response to ursodeoxycholic acid.

### EXAMPLES

### NCT04526665 Clinical Trial (ELATIVE™)

Participants aged 18 to 75 years, who had been diagnosed with primary biliary cholangitis according to established criteria, were recruited at 82 sites in 14 countries. Inclusion criteria were an alkaline phosphatase level of ≥1.67 times the upper limit of the normal range (ULN), and a total bilirubin level of ≤2 times the ULN. Prior to screening, eligible participants had received 13 to 15 mg/kg ursodeoxycholic acid treatment per day for at least 12 months (stable dose for at least 3 months) or were intolerant to ursodeoxycholic acid (no treatment for at least 3 months).

In this double-blind, placebo-controlled, phase 3 trial, participants with an inadequate response or intolerance to ursodeoxycholic acid were randomized 2:1 to receive elafibranor 80 mg or placebo once daily. Participants receiving stable doses of ursodeoxycholic acid for at least three months prior to enrollment continued their treatment regimen throughout the trial. The overall double-blind treatment period consisted of two parts. In part one, all randomized patients received a minimum of 52 weeks of double-blind treatment. In part two, participants continued to receive double-blind treatment beyond Week 52 in a variable treatment period until all participants had completed their Week 52 assessment or until a maximum treatment duration of 104 weeks, whichever came first. The first database lock occurred after the last participant completed its Week 52 visit in the double-blind period. At the end of the double-blind period (lasting 52 to 104 weeks), participants could optionally enter an open-label extension where they would receive elafibranor 80 mg for up to five years.

From September 2020 through May 2023, 161 participants were randomly assigned to receive elafibranor 80 mg (108 participants) or placebo (53 participants); these participants made up the intent-to-treat (ITT) and safety populations. The pruritus intent-to-treat population included 66 participants with moderate-to-severe pruritus (44 receiving elafibranor, 22 receiving placebo).

As fatigue is a major symptom of PBC, it is important to ensure that the scales correctly capture the participants' full experience of symptoms during clinical studies and adequately report the impact of each symptom.

### PROMIS Fatigue Short Form (SF) 7a Questionnaire

The Patient Reported Outcome Measurement Information System (PROMIS®) is the product of a massive effort begun in 2004 and based on modern measurement theory, to address the need to develop precise, consensus measures of health outcome, including fatigue, suitable for use across chronic diseases in particular PBC. The PROMIS Fatigue Short Form 7a (PROMIS Fatigue SF 7a) is a seven-item questionnaire derived from the 95-item PROMIS Fatigue item bank.

The PROMIS Fatigue item banks assess a range of self-reported symptoms, from mild subjective feelings of tiredness to an overwhelming, debilitating, and sustained sense of exhaustion that likely decreases one's ability to execute daily activities and function normally in family or social roles. Fatigue is divided into the experience of fatigue (frequency, duration, and intensity) and the impact of fatigue on physical, mental, and social activities. The fatigue short forms are universal rather than disease-specific. All assess fatigue over the past seven days.

The PROMIS Fatigue SF 7a (7-items) measures both the experience of fatigue and interference of fatigue on daily activities over the past 7 days using a Likert scale. PROMIS Fatigue SF 7a includes seven items with response options on a 5-point Likert scale, ranging from 1=“never” to 5=“always.” One item, “How often did you have enough energy to exercise strenuously,” is reverse scored so that higher scores indicate greater fatigue.

Participants reported outcome measures of fatigue evaluated in Study NCT04526665 included the PROMIS Fatigue SF 7a questionnaire.

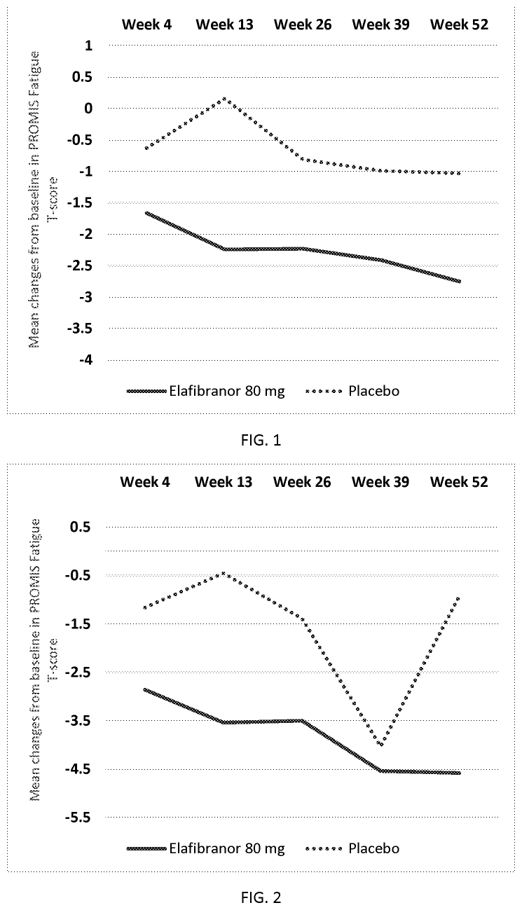

The analyses were performed in both the ITT (ITT=intent-to-treat) and the Pruritus ITT Analyses Sets (see Table 1, Table 2, Table 3, and FIG. 1 and FIG. 2).

In the Pruritus ITT Analysis Set, participants with a higher baseline PBC Worst Itch Numeric Rating Scale (WI-NRS) score also had a higher baseline PROMIS Fatigue T-score, and showed a greater reduction from baseline at Week 52 (implying improvement) on elafibranor 80 mg compared to placebo as measured by the PROMIS Fatigue T-score with a LS mean difference from placebo of −3.77 ([95% CI: −8.01; 0.47]: p=0.0802). Such a reduction was already observed at Week 13 with a LS mean difference from placebo of −3.15 ([95% CI: −6.62; 0.33]: p=0.0752).

In the ITT Analysis Set, a greater reduction from baseline was observed in participants treated with elafibranor 80 mg compared to placebo at Week 13 (LS mean difference from placebo of −2.11 [95% CI: 4.21; 0.01]: p=0.0494), than at Week 52 (LS mean difference from placebo of −1.23 [95% CI: (−3.64; 1.18]: p=0.3161).

TABLE 1
Change from Baseline in PROMIS Fatigue
T-score at Week 13 - MMRM

| | Analysis Set | | | |
|---|---|---|---|---|
| | ITT | | Pruritus ITT | |
| Analysis Visit | Elafibranor | | Elafibranor | |
| Observed/Change | 80 mg | Placebo | 80 mg | Placebo |
| Statistic | N = 108 | N = 53 | N = 44 | N = 22 |
| Baseline, n | 107 | 52 | 43 | 21 |
| Mean | 55.86 | 54.70 | 60.88 | 61.68 |
| Median | 56.40 | 56.40 | 63.40 | 62.00 |
| Week 13, n | 104 | 48 | 43 | 18 |
| Mean | 53.63 | 54.35 | 57.49 | 60.34 |
| Median | 52.95 | 54.40 | 59.20 | 59.90 |
| Change from baseline, n | 103 | 47 | 42 | 17 |
| Mean | −2.24 | 0.16 | −3.54 | −0.45 |
| Median | −1.50 | 0 | −2,35 | 0 |
| LS means (95% CI) | −2.16 | −0.05 | −3.50 | −0.35 |
| LS means difference with | −2.11 | | −3.15 | |
| placebo (95% CI) | [−3.36; −0.96] | | [−6.62; 0.33] | |
| p-value | 0.0494 | | 0.0752 | |

TABLE 2
Change from Baseline in PROMIS Fatigue
T-score at Week 52 - MMRM

| | Analysis Set | | | |
|---|---|---|---|---|
| | ITT | | Pruritus ITT | |
| Analysis Visit | Elafibranor | | Elafibranor | |
| Observed/Change | 80 mg | Placebo | 80 mg | Placebo |
| Statistic | N = 108 | N = 53 | N = 44 | N = 22 |
| Baseline, n | 107 | 52 | 43 | 21 |
| Mean | 55.86 | 54.70 | 60.88 | 61.68 |
| Median | 56.40 | 56.40 | 63.40) | 62.00 |
| Week 52, n | 95 | 47 | 42 | 17 |
| Mean | 53.70 | 53.14 | 56.23 | 60.14 |
| Median | 53.70 | 52.20 | 56.45 | 62.00 |
| Change from baseline, n | 95 | 46 | 42 | 16 |
| Mean | −2.75 | −1.03 | −4.58 | −0.93 |
| Median | −2.80 | 0 | −4.20 | 0 |
| LS means (95% CI) | −2.45 | −1.23 | −4.53 | −0.76 |
| LS means difference with | −1.23 | | −3.77 | |
| placebo (95% CI) | [−3.64; 1.18] | | [−8.01; 0.47] | |
| p-value | 0.3161 | | 0.0802 | |

CI = confidence interval; ITT = intent-to-treat; LS = least squares; Max = maximum; Min = minimum; MMRM = mixed model for repeated measures; N = total number of participants in a group; n = number of participants with non-missing data; PROMIS = Patient Reported Outcome Measurement Information System.

Baseline is defined as the last non-missing value on or before the first dose of the randomised study medication.

TABLE 3
Change from Baseline in PROMIS Fatigue T-score overtime
(from Week 4 to Week 52)
See FIG. 1 and FIG. 2

| | Analysis Set | | | |
|---|---|---|---|---|
| | ITT | | Pruritus ITT | |
| Analysis Visit | Elafibranor | | Elafibranor | |
| Observed/Change | 80 mg | Placebo | 80 mg | Placebo |
| Statistic | N = 108 | N = 53 | N = 44 | N = 22 |
| Baseline, n | 107 | 52 | 43 | 21 |
| Mean score | 55.86 | 54.70 | 60.88 | 61.68 |
| Median score | 56.40 | 56.40 | 63.40 | 62.00 |
| Week 4, n | 106 | 51 | 44 | 21 |
| Change from baseline, n | 105 | 50 | 44 | 20 |
| Mean | −1.66 | −0.63 | −2.86 | −1.16 |
| Median | −1.60 | 0.00 | −2.70 | −0.85 |
| Week 13, n | 104 | 48 | 43 | 18 |
| Change from baseline, n | 103 | 47 | 42 | 17 |
| Mean | −2.24 | 0.16 | −3.54 | −0.45 |
| Median | −1.50 | 0.00 | −2.35 | 0.00 |
| Week 26, n | 104 | 46 | 43 | 16 |
| Change from baseline, n | 104 | 45 | 43 | 15 |
| Mean | −2.23 | −0.81 | −3.50 | −1.39 |
| Median | −1.55 | 0.00 | −3.20 | −2.80 |
| Week 39, n | 100 | 46 | 42 | 17 |
| Change from baseline, n | 100 | 45 | 42 | 16 |
| Mean | −2.41 | −0.99 | −4.54 | −4.03 |
| Median | −1.45 | 0.00 | −4.05 | −5.65 |
| Week 52, n | 95 | 47 | 42 | 17 |
| Change from baseline, n | 95 | 46 | 42 | 16 |
| Mean | −2.75 | −1.03 | −4.58 | −0.93 |
| Median | −2.80 | 0.00 | −4.20 | 0.00 |

Treatment with elafibranor resulted in an improvement in fatigue as measured by the PROMIS Fatigue SF 7a questionnaire compared to placebo. This improvement was better in patients with moderate to severe pruritus at baseline, proving treatment with elafibranor provides important symptom relief for participants with PBC and pruritus.

1. A method for reducing fatigue in a subject having primary biliary cholangitis (PBC), the method comprising administering to said subject a therapeutically effective amount of a pharmaceutical composition comprising a compound selected from elafibranor, 2-[2,6-dimethyl-4-[3-[4-(methylthio)phenyl]-3-oxo-propyl]phenoxy]-2-methylpropanoic acid (GFT1007), and a pharmaceutically acceptable salt of elafibranor or GFT1007.

2. The method of claim 1, wherein the subject having PBC also has pruritus.

3. The method of claim 1, wherein the therapeutically effective amount of the pharmaceutical composition reduces a PROMIS Fatigue T-score by at least 1 point from baseline in the subject.

4. The method of claim 2, wherein the therapeutically effective amount of the pharmaceutical composition reduces a PROMIS Fatigue T-score by at least 2 points from baseline in the subject having PBC and pruritus.

5. The method of claim 1, wherein the pharmaceutical composition comprises elafibranor or the pharmaceutically acceptable salt thereof.

6. The method of claim 5, wherein the pharmaceutical composition comprises elafibranor.

7. The method of claim 1, wherein the pharmaceutical composition is formulated in a form selected from the group consisting of a gel, an oil, a pill, a tablet, a powder, a gel cap, a capsule, and a galenic form or device assuring a prolonged and/or slow release.

8. The method of claim 7, wherein the pharmaceutical composition is formulated in the form of a tablet.

9. The method of claim 6, wherein the pharmaceutical composition is administered once a day.

10. The method of claim 9, wherein the administration is oral.

11. The method of claim 1, wherein the therapeutically effective amount of the pharmaceutical composition contains 80 mg or 120 mg of the compound per administration.

12. The method of claim 11, wherein the therapeutically effective amount of the pharmaceutical composition contains 80 mg of elafibranor per administration.

13. The method of claim 1, wherein the subject has an inadequate response to ursodeoxycholic acid (UDCA).

14. The method of claim 1, further comprising administering ursodeoxycholic acid (UDCA) to the subject.

15. The method of claim 14, wherein the pharmaceutical composition comprises elafibranor at a dose of 80 mg per administration.

16. The method of claim 15, wherein the subject has an inadequate response to UDCA.

17. A method for treating PBC in a subject having PBC with fatigue without provoking or worsening the fatigue in the subject, the method comprising administering to the subject a therapeutically effective amount of a pharmaceutical composition comprising a compound selected from the group consisting of elafibranor, GFT1007, and a pharmaceutically acceptable salt of elafibranor or GFT1007.

18. A method for reducing fatigue associated with PBC in a subject having PBC, the method comprising administering to said subject a therapeutically effective amount of a pharmaceutical composition comprising a compound selected from the group consisting of elafibranor, GFT1007, and a pharmaceutically acceptable salt of elafibranor or GFT1007.
